(19) United States
(12) Patent Application Publication
(10) Pub. No.: US 2019/0241847 A1
(43) Pub. Date: Aug. 8, 2019

(54) SYMBIOTIC ALGAE SYSTEM

(71) Applicant: GSR Solutions LLC, Burlington, VT (US)

(72) Inventor: Anju D. Krivov, Burlington, VT (US)

(21) Appl. No.: 16/387,163

(22) Filed: Apr. 17, 2019

Related U.S. Application Data
(63) Continuation-in-part of application No. 15/856,805, filed on Dec. 28, 2017.
Application No. 15/856,805 is a division of application No. 14/932,218, filed on Nov. 4, 2015.
Application No. 14/932,218 is a continuation-in-part of application No. 14/888,986, filed on Nov. 4, 2015.
Application No. 14/888,986 is a continuation-in-part of application No. PCT/US18/67972, filed on Dec. 28, 2018.
Application No. PCT/US18/67972 is a continuation of application No. 15/856,642, filed on Dec. 28, 2017.
(60) Provisional application No. 62/067,049, filed on Oct. 22, 2014.
(60) Provisional application No. 62/067,042, filed on Oct. 22, 2014.
(60) Provisional application No. 62/079,135, filed on Nov. 13, 2014.

(51) Int. Cl. C12M 1/00; C12M 1/26; C02F 3/32; C02F 11/04; C12M 1/12

(52) U.S. Cl. CPC C12M 21/02; C12M 29/00; C12M 33/00; C02F 3/322; C12M 29/14; C12M 29/18; C02F 2101/103; C02F 3/325; C12M 47/10; C12M 25/00; C12M 29/24; C12M 43/00; C02F 11/04

(57) ABSTRACT

According to present disclosure, there is disclosed an algae growth and cultivation system that provides a cost-efficient means of producing algae biomass as feedstock for algae-based products, such as, biofuel manufacture, and desirably impacts alternative/renewable energy production, nutrient recovery from waste streams, and valued byproducts production. The system as discussed herein is an integrated systems approach to wastewater treatment, algal strains selection for byproducts production, and recycle of algal-oil extraction waste or additional algae harvested as feedstock for fertilizer production. Embodiments of a system as discussed herein present an economically viable algae production system and process that allows algae-derived products such as biofuels, fertilizer, etc. to compete with petroleum products in the marketplace.

### RELATED APPLICATION DATA

This application is a continuation-in-part application of U.S. patent application Ser. No. 15/856,805, filed Dec. 28, 2017, entitled “Symbiotic Algae System,” which is a divisional of U.S. patent application Ser. No. 14/932,218, filed Nov. 4, 2015, and entitled “Symbiotic Algae System”, which is a continuation-in-part of U.S. patent application Ser. No. 14/888,986, filed Nov. 4, 2015, entitled “Symbiotic Algae System with Looped Reactor,”, which is a national stage entry of PCT Application No. PCT/US15/56344, filed on Oct. 20, 2015, and entitled “Symbiotic Algae System with Looped Reactor,” which claims priority to U.S. provisional application Ser. No. 62/067,049, filed Oct. 22, 2014, and entitled “Symbiotic Algae System with Looped Reactor,” U.S. provisional application Ser. No. 62/067,042, filed Oct. 22, 2014, and entitled “Symbiotic Algae System,” and U.S. provisional application Ser. No. 62/079,135, filed Nov. 13, 2014, and entitled “Algal Growth System Process Utilizing Intermediate Products of Consolidated Bioprocessing Process or Anaerobic Digestion Process,” each of the aforementioned applications are hereby incorporated by reference herein in their entirety. This application is also a continuation-in-part of PCT Application No. PCT/US2018/067972, filed Dec. 28, 2018 and entitled “Systems and Methods of Producing Compositions from the Nutrients Recovered from Waste Streams,” which claims priority to U.S. patent application Ser. No. 15/856,642, filed Dec. 28, 2017 and entitled “Systems and Methods of Producing Compositions from the Nutrients Recovered from Waste Streams,” each of the aforementioned applications are hereby incorporated by reference herein in their entirety.

### FIELD OF THE INVENTION

The present invention relates to algae growth systems and in particular to a Symbiotic Algae System.

### BACKGROUND

Mass cultivation of algae has been used for creating nutritional supplements, fertilizer, and food additives. Commercial growth of algae has also been explored to create biologically-derived energy products such as biodiesel, bioethanol, and hydrogen gas. As a biofuel feedstock, algae provide multiple environmental benefits and present significant advantages over traditional plants/crops used for biofuel production (e.g., corn, sugarcane, switch-grass, etc.). For example, unlike traditional food crops that are being used to produce biofuels (e.g., corn, sugarcane, etc.), algae does not compete with food and water resources; it grows significantly faster than traditional crops used for biodiesel; algae produce up to 300 times more oil than traditional crops on an area basis; algae fuel has properties (low temperature and high energy density) that make it suitable as jet fuel; and algae can be produced so as to provide a nearly continuous supply of fuel. Moreover, algae can treat industrial, municipal, and agricultural wastewaters, capture carbon-dioxide, and provide valuable byproducts, such as, but not limited to, protein-rich feed for farm animals, organic fertilizer, and feedstock for producing biogas.

Algal biomass can accumulate up to 50% carbon by dry weight, therefore producing 100 tons of algal biomass fixes roughly 183 tons of CO2—providing a tremendous potential to capture CO2 emissions from power plant flue gases and other fixed sources. Ideally, biodiesel from algae can be carbon neutral, because all the power needed for producing and processing the algae could potentially come from algal biodiesel and from methane produced by anaerobic digestion of the biomass residue left behind after the oil has been extracted.

The successful role of algae in wastewater treatment has been documented since the early 1950s, and algal wastewater treatment systems are known to utilize the extra nutrients including nitrogen, phosphorus, potassium, heavy metals, and other organic compounds from wastewater. For example, an algal turf scrubber system feeding algae a diet of dairy manure can recover over 95% of the nitrogen and phosphorus in the manure wastewater. Additionally, lipid/oil productivity occurs in algal wastewater treatment systems, but there are few, if any, known robust algae strain(s) for oil production that use wastewater as a primary feedstock. For example, a polyculture (dominated by Rhizoclonium sp.) used in algal turf systems for treating dairy and swine wastewater had very low lipids/oil content (fatty acids contents of 0.6% to 1.5% of dry algae weight) and other researchers have reported 2.8 g/m² per day of lipid productivity from algal polyculture combined with dairy wastewater treatment.

Algae's other byproducts can also be beneficial. For example, the value of algae as food was explored as early as 1950s, and some have demonstrated the concept by raising baby chickens to adults on twenty percent (20%) algae fortified feed (grown on pasteurized chicken manure). The antibiotic Chlorellin extracted from Chlorella during World War II marked the start of an algae based pharmaceutical and nutraceutical industry that led to the Japanese Chlorella production facilities during 1960s, further leading to current production of Chlorella, Spirulina, Dunaliella and Hematococus on a commercial scale. Fertilizers from algae have also shown equivalence to commercial organic fertilizers in terms of plant mass and nutrient content.

Despite all of the aforementioned benefits, algae biomass production and the production of algal oil (i.e., biofuels from algae) are primarily hampered by the high cost of producing algae biomass (currently either requiring large amounts of land/water and/or large sterile facilities). There have been attempts to offset this high cost by using the various traits of algae to their greatest benefit. For example, biofuel production from algae has been combined with wastewater treatment (as discussed above) and has been shown to be 40% more cost effective than the best conventional alternatives, but still has not been economically viable due to low lipid production. As another example, entities have attempted to vary the type of cultures used—for example, algae monoculture (requiring sterile conditions) versus polyculture-based wastewater treatment. However, the results of these trials have not proven themselves. Other disadvantages of current algae biomass production include, but are not limited to, the availability of low-cost throughput sugar feed-stocks for growing algae, treating effluent created during production, and the requirement of nitrogen and phosphorus supplements. Until such time as these algae production related issues are solved, production of oil feedstock from algae is likely to remain commercially infeasible.

For this reason, the system and process disclosed herein addresses the challenges involved in materializing cost-efficient algae based on a robust, easily adaptable, environmentally friendly system that is capable of growing algae biomass on a commercial scale for biofuel, fertilizer, animal feed, and other byproducts. The symbiotic algae system and process disclosed herein also holds great potential for industries, farms and municipalities, especially dairy farms and breweries, because the system allows these entities to more efficiently and effectively meet government standards for handling and recycling of wastes.

### SUMMARY

In an exemplary aspect, a symbiotic algae system comprises a first algal growth component, wherein the first algal growth component includes a heterotrophic organism, and wherein the first algal growth component produces a first effluent and an off-gas; and a second algal growth component is fluidly coupled to the first algal growth component, and the second algal growth component including at least one organism from the group of: a photoautotrophic organism, a mixotrophic organism, and a heterotrophic organism, and wherein the second algal growth component receives, as an input, the first effluent and the off-gas and produces a second effluent.

In another exemplary aspect, a symbiotic algae system comprises a first algal growth component, wherein the first algal growth component includes a heterotrophic organism, and wherein the first algal growth component produces an first effluent and an off-gas; and a second algal growth component fluidly coupled to the first algal growth component, wherein the second algal growth component includes at least one organism from the group of: a photoautotrophic organism, a mixotrophic organism, and a heterotrophic organism, and wherein the second algal growth component receives, as a first input, the first effluent and the first off-gas and produces an second effluent and a second off-gas; and wherein the second effluent and the second off-gas are received as inputs to the first algal growth component.

In yet another exemplary aspect, a symbiotic algae system comprises a waste nutrient preparation sub-system; an algal culturing system including: a first algal growth component fluidly coupled to the waste-nutrient preparation sub-system, wherein the first algal growth component includes a heterotrophic organism, and wherein the first algal growth component produces a first effluent and an off-gas; and a second algal growth component, wherein the second algal growth component includes at least one organism from the group of: a photoautotrophic organism, a mixotrophic organism, and a heterotrophic organism, and wherein the second algal growth component receives, as an input, the effluent and the off-gas and produces a second effluent; and an algal harvesting system fluidly coupled to the algal culturing system; an algal biomass processing system fluidly coupled to the algal harvesting system; and a byproducts system fluidly coupled to the algal biomass processing system.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary symbiotic algae system according to an embodiment of the present invention;

FIG. 2 is a block diagram of an algal core suitable for use with an exemplary symbiotic algae system such as the systems shown in FIGS. 1 and 5;

FIG. 3 is a block diagram of another algal core suitable for use with an exemplary symbiotic algae system such as the systems shown in FIGS. 1 and 5;

FIG. 4 is a block diagram of another algal core suitable for use with an exemplary symbiotic algae system such as the systems shown in FIGS. 1 and 5;

FIG. 5 is a block diagram of a portion of an exemplary symbiotic algae system according to another embodiment of the present invention;

FIG. 6 is a chart of algal cell density showing the optical density over time for a test core according to an embodiment of the present invention and a control;

FIG. 7 is a block diagram of a portion of an exemplary symbiotic algae system suitable for removing contaminants according to another embodiment of the present invention

FIG. 8 is a block diagram of an exemplary process of removing contaminants from a waste stream according to an embodiment of the present invention;

FIG. 9 is a block diagram of a process of pairing an algal strain to a given effluent according to an embodiment of the present invention; and

FIG. 10 is a table showing prior art energy returns for biodiesel using various feed-stocks.

### DETAILED DESCRIPTION

A symbiotic algae system according to the present disclosure provides a cost-efficient means of producing algae biomass for many applications, such as, but not limited to, feedstock for biofuel manufacture which desirably impacts alternative/renewable energy production, nutrient recovery from waste streams, and valued byproducts production (nutraceuticals, pharmaceuticals, animal feed, etc.). A symbiotic algae system as discussed herein is an integrated systems approach to wastewater treatment, algal strains selection for oil production, CO2 capture or nutrient capture from heterotrophic processes, and recycle of algal-oil extraction waste as feedstock for biogas production. Embodiments of a symbiotic algae system as discussed herein present an economically viable algae production system and process that allows algae-derived biofuels to compete with petroleum products in the marketplace.

A symbiotic algae system as discussed herein is, at a high level, a scalable process for cultivating algae biomass, in which a heterotrophic (i.e., non-light dependent) algal growth strain is used to provide carbon dioxide and/or effluent to a photoautotrophic or mixotrophic or a combination of the three cultivation processes (i.e., photoautotrophic, mixotrophic, and heterotrophic) while concomitantly producing algae biomass or lipids for biofuel production. In certain embodiments, the photoautotrophic or mixotrophic or heterotrophic cultivation portion of the symbiotic algae system may result in the cultivation of additional algae biomass, but could include (alternatively or additionally) the cultivation of any photoautotrophically or mixotrophically grown microbial plant matter that requires carbon dioxide and/or effluent containing nutrients, such as nitrogen, phosphorus, and organic carbon. As will be discussed in more detail below, the symbiotic algae system can efficiently use nutrients from both commercial and/or other waste streams for the production of lipids for use with biofuels, and as such, the energy return on investment scenarios are significantly higher than previously considered possible. This symbiotic algae system provides a robust scalable option which has improved cost efficiencies due to production of additional desirable byproducts such as fertilizer.

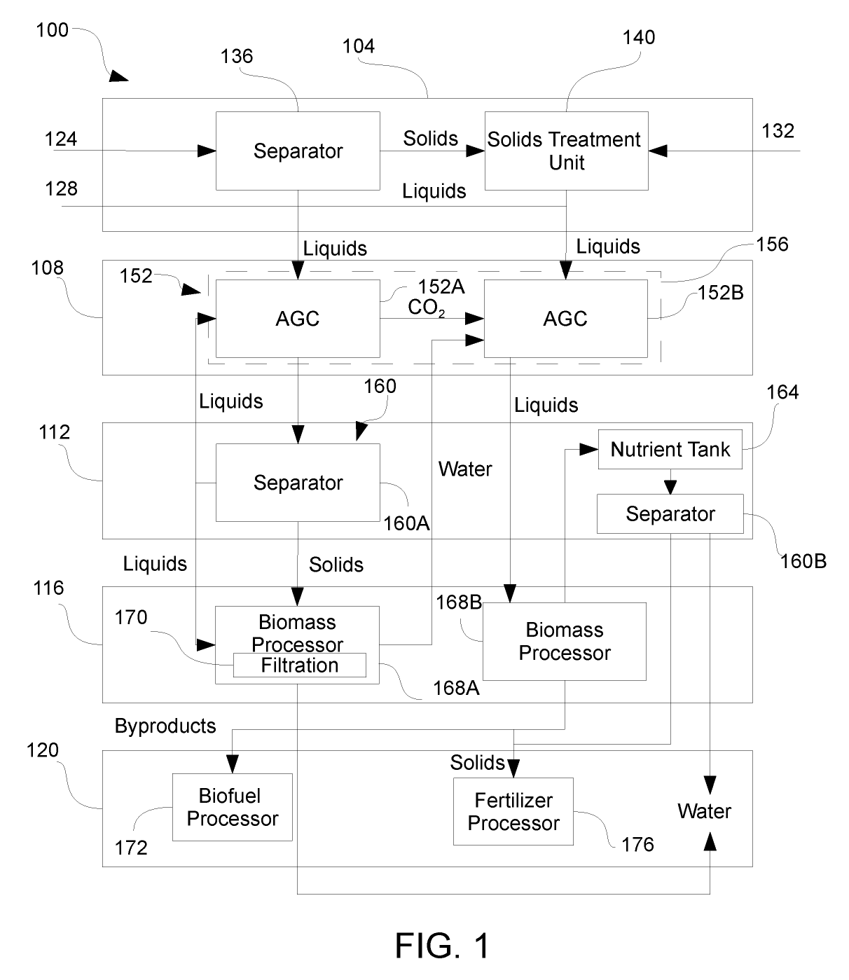

Turning now to the figures, and specifically with reference to FIG. 1, there is shown a symbiotic algae system (SAS) 100. In an exemplary embodiment, SAS 100 includes, at a high level, a waste nutrient preparation sub-system 104, an algal culturing system 108, an algal harvesting system 112, an algal biomass processing system 116, and a byproducts system 120.

Waste nutrient preparation sub-system 104 is generally configured to treat incoming feedstocks (e.g., manure, municipal waste) for the rest of SAS 100. The design and configuration of waste nutrient preparation sub-system 104 depends on the desired inputs for SAS 100. As shown in FIG. 1, waste nutrient sub-system 104 includes three inputs: an effluent input 124, a fresh water input 128, and a waste input 132. Effluent input 124 can generally be any nutrient rich liquid waste before or after single or multiple pre-treatments, for example, dairy farm effluent, agricultural wastewater streams, brewery liquid waste streams, municipal waste, food waste, digested effluent etc. Effluent input 124 is fed into a separator 136 that separates the effluent solids and liquids, using methods such as settling, filtration, or via centrifugal separators. The solids can then be fed to a solids treatment unit 140, such as a digester, which can, among other things, break down the solids into a feed stream suitable for further use within SAS 100, such as a source of carbon dioxide and sugars, or into other byproducts (e.g., biogas, fertilizers, etc.). Solids treatment unit 140 can also accept waste input 132 for processing solids. The output of solids treatment unit 140 and the liquid effluent separated by separator 136 may be combined with fresh water input 128 to prepare the feedstock for algal culturing system 108 that includes one or more algae growth components (AGC) 152, e.g., AGC 152A and AGC 152B. In another embodiment, waste input 132 can first be processed by solids treatment unit 140 and then be separator 136.

In an exemplary embodiment, waste nutrient preparation sub-system 104 is a manure settling and solids preparation unit that outputs liquid manure waste to algal culturing system 108. In this embodiment, manure is combined with water run-off (e.g., fresh water input 128) and collected in a large separation tank (e.g., separator 136). The denser solids are allowed to sink to the bottom (or in certain embodiments are mechanically separated) and the output liquid manure water is pumped from the tank. In an exemplary embodiment, solid wastes, for example, ligno-cellulosic material such as grain spoilage or grasses, is pretreated in solids treatment unit 140 with or without manure effluent to prepare the nutrients (e.g., different forms of nitrogen or phosphorus or sugars or organic carbon) for algal culturing in algal culturing system 108.

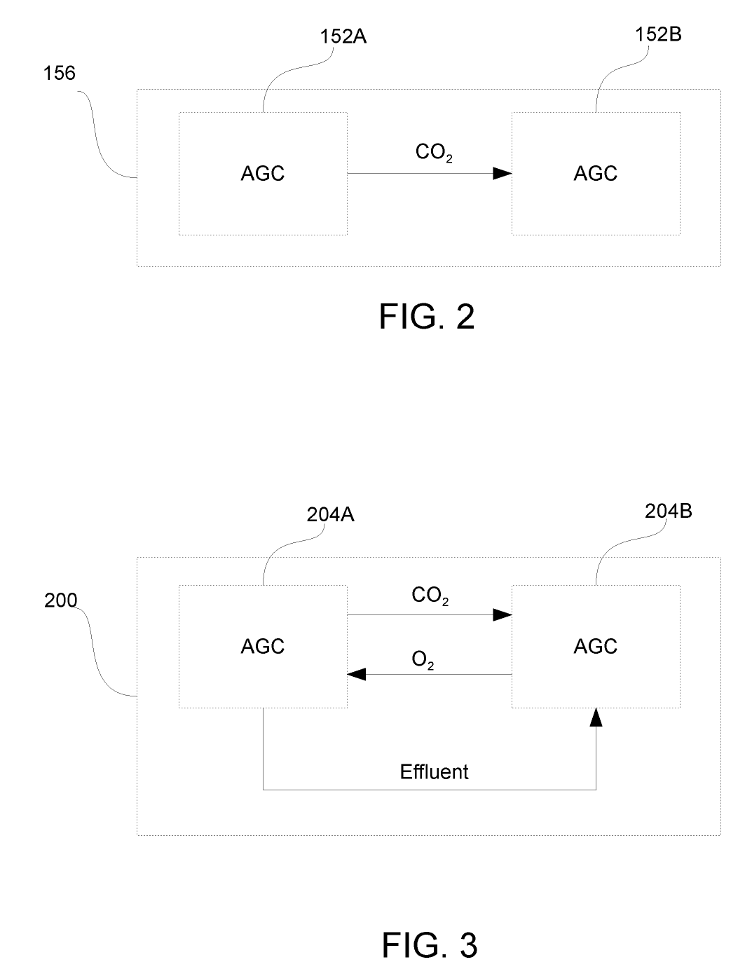

Algal culturing system 108 is generally configured to grow algal biomass from numerous nutrient and/or waste streams. In an exemplary embodiment, algal culturing system includes an algal core 156 (FIG. 2), which can include an AGC 152A that is coupled to, and mutually supports, an AGC 152B. In an exemplary embodiment, AGC 152A is an organic carbon source fed heterotrophic algae and AGC 152B is one or more of a photoautotrophic, mixotrophic, and heterotrophic algae. In general, heterotrophic algal production produces higher amounts of oil/lipids compared to its lighted dependent counterpart (e.g., mixotrophic, photoautotrophic); however, it is limited in its ability to capture nutrients or other desired extracts (lipids, certain proteins, carbohydrates, metabolites, dyes, bioplastics, etc.) and also generates effluent that typically requires treatment. Algal culturing system 108 combines the two complementary approaches thereby providing a system that can produce high amounts of oil/lipids and can capture nutrients for byproducts such as fertilizer production that can offset the costs of algal biomass production. For example, the algae Chlorella vulgaris can remove up to about 20.8% of phosphate under autotrophic conditions, up to about 17.8% under heterotrophic conditions, and up to about 20.9% under mixotrophic conditions after 5 days when grown in synthetic wastewater. Algal culturing system 108, in certain embodiments described herein, can capture the remaining nutrients left after the heterotrophic algal growth stage and recycles these nutrients for the autotrophic/mixotrophic algal growth and vice versa. Additionally, algal culturing system 108 can also be designed to recycle the CO2 produced as a result of heterotrophic mode of algal growth to the autotrophic/mixotrophic growth, and can recycle the oxygen produced by the autotrophic/mixotrophic growth for heterotrophic growth. The recycling of nutrients for different trophic growth provides additional cost offsets made possible via algal culturing system 108.

As discussed in more detail below, the design of algal core 156 determines the amount of algae produced in AGC 152B based on the amount of CO2 produced by AGC 152A or vice versa with oxygen production by AGC 152A fed to AGC 152B. For example, if AGC 152A produces about 1.8 tons of CO2, one would expect that up to about 1 ton of dry algae biomass would be produced by AGC 152B.

AGC 152A has the advantage of accepting a myriad of inputs. For example, and as described previously, AGC 152A can use liquid manure waste as in input, or can use organic carbon from commercially available clean sources (e.g. sugars) or other waste streams, such as, but not limited to, grains spoilage from farms, brewery waste, liquids containing sugars from food waste, industrial wastes, farm operation wastes, or a mixture of different wastes. Algal biomass production at AGC 152A can be maximized by using the naturally occurring or genetically enhanced algae strains, monoculture or polyculture, and/or other microbial strains such as bacteria and/or fungi that is best suited for the feedstock (e.g. sugars available from market or from waste sources) available at the target location. In other words, certain algae do better with certain carbon inputs than others and the determination of which algal strain should/can be used may be based on a number of factors, including, but not limited to, the input stream, desired output (e.g., a certain reduction in a certain component of the input steam or to maximize the growth of biomass), expected temperature, and pH. In an exemplary embodiment, the algae, Chlorella vulgaris, has been successfully cultured in dairy manure effluents. In another embodiment, AGC 152A can use and produce non-algae strains, such as the fungal strain, Trichoderma reesei, for converting aforementioned throughput feedstock into byproducts.

In an exemplary embodiment, AGC 152A includes heterotrophic algae, which is known to produce dense algae growth and a relatively high amount of useful byproducts. Heterotrophic algae can be grown in containers such as fermenter(s), or closed or open system(s), or a combination or a hybrid form of the aforementioned. Standalone growth of heterotrophic algae is scalable in large sized vessels (such as, but not limited to, fermenters), and under heterotrophic growth conditions, respiration rates equal or exceed the theoretical minimum cost of biomass synthesis and biomass synthesis can achieve nearly the maximal theoretical efficiency.

One of the outputs (in addition to generated algal biomass for lipid extraction) of AGC 152A is an off-gas, CO2, which is generated as a result of algae respiration due to organic uptake of carbon. The CO2 generated by AGC 152A is used as an input for AGC 152B.

AGC 152B is designed to accept the output of AGC 152A (which are typically byproducts of AGC 152A, such as, but not limited to, suspended solids, inorganic carbon, organic carbon, phosphorus, potassium, nitrogen (in various forms including ammonium, nitrate nitrogen, and suspended nitrogen)). As such, AGC 152B can be a photoautotrophic, a mixotrophic, or a combination of both photoautotrophic and mixotrophic production systems of algae fed by the CO2 produced by AGC 152A. AGC 152B can take the form of open, closed, or hybrid systems of algae growth and therefore can be implemented by various methodologies, such as, but not limited to, a tank, a bag, a fermenter, a tubular vessel, a plate, and a raceway, of any shape, size, or volume. Typically, AGC 152A and 152B are in separate containers or growing areas as it facilitates the independent growth of each algae in each AGC.

In an exemplary embodiment, AGC 152B uses clean sources of additional nutrients or captures nutrients from waste or wastewater streams, for example, but not limited to, anaerobically or aerobically digested effluent from dairy farms, industrial operations such as breweries, food waste, municipal waste, etc. The CO2 input stream from various industrial operations, such as flue gases, supplied to second algal growth component 312 may contain other nutrients that promote algae biomass growth. While AGC 152B has been previously described as one or more of a photoautotrophic, mixotrophic, and heterotrophic algal growth, it could also include the cultivation of any biomass that requires the addition of inorganic carbon (CO2) and/or organic carbon and/or nutrients (such as nitrogen and phosphorus and other micro or macro nutrients) for its growth.

In order to size algal core 156 (and ultimately determine an estimate of the total expected biomass (TEB) production of the system), the amount of algal biomass producible from AGC 152A at the site is determined based on the amount and type of throughput feedstock available, e.g., the amount available from on-site sources, brought from off-site sources, or combination of the two, to grow the respective algae type used in AGC 152A. For example, if the feedstock is nitrogen rich, algal types that may be paired with this feedstock include Chlorella vulgaris, Chlamydomonas reinhardtii, and Scenedesmus abundans. Alternatively, if the feedstock is phosphate rich, the algal types that may be paired with this feedstock include the bacteria Acinetobacter calcoaceticus or Acinetobacter johnsonii. Based upon the expected algal biomass producible from AGC 152A, an amount of CO2 available to AGC 152B from AGC 152A can be determined. The available CO2 and the amount of feedstock available to AGC 152B is determinative of the amount of biomass producible of AGC 152B. The TEB can then be determined as the sum of the algal biomass produced at AGC 152A and the biomass produced at AGC 152B.

The amount of biomass producible by either growth component, i.e., AGC 152A and AGC 152B, will be heavily influenced by the specific algae chosen for each respective component, and in the case of AGC 152B, the type of algae chosen. For example, a mixotrophic algal growth system requires less CO2 because it requires greater organic carbon uptake when compared to a phototrophic system. The type of algal system chosen for AGC 152B (and the specific algae) can be used to determine the size or volume required for AGC 152B when implemented in the form of, for example, a closed photobioreactor, an open tank, a raceway, or a pond system. For example, if an output of 1000 tons of Chlorella vulgaris grown in AGC 152B (e.g. a photobioreactor), that system would need at least 1800 tons CO2 as an off-gas input. To produce that much CO2, AGC 152A would need to be sized to grow enough heterotrophic biomass. In contrast, in the case of mixotrophic algal production, the CO2 requirement could be about 10 times lower.

A non-limiting example of the setup of algal culturing system 108 and the selection process for the algal strains used therein is now presented. In this example, the effluent or input into AGC 152 is brewery waste. This waste has a composition as follows:

Suspended solids (TSS) (mg/L) 318.00

Total C of TSS % dry wt. 18.50

Total N mg/L 14.74

NH4-N mg/L mg/L 0.05

Ortho-Phosphate mg/L 21.20Total P mg/L 23.60

K mg/L 53.90

Ca mg/L 33.40

Mg mg/L 10.30

Na mg/L 202

Fe mg/L 0.60

Zn mg/L 0.11

S mg/L 43.6

Based on literature and market research, and followed up by small scale experimentation, the strain Chlorella was chosen for AGC 152A due to its ability to use high amounts organic carbon (20% of dry weight of suspended solids is in the input stream) and because it also requires some nitrogen (N), phosphorus (P), and potassium (K) to grow. Chlorella also does not produce any toxic byproducts during growth that would need to be considered when selecting the algal strain for AGC 152B. Thus, given the composition of the input stream and the growth characteristics of Chlorella, it was selected to grow in a heterotrophic environment. The conditions for growing Chlorella included a temperature range of 28-32° C. and pH 6.5-7—these conditions were monitored using known techniques and adjustments to temperature and pH were made as necessary to support the algae's growth. Additional considerations for the heterotrophic system can include, but are not limited to, the oxygen level, the dissolved CO2 level, and the concentration of solids, each of which would impact growth rates, but not necessarily impact the choice of algae strain. It should be noted that other strains would also be suitable for growth with this input stream in a heterotrophic environment and a different strain may be chosen depending on whether the output from the second algal growth system is added as an input to the first algal growth system (and thus adding additional materials). In some embodiments, a systems solution may be necessary (e.g., an iterative analysis that evaluates a system as a whole to understand the impact of an alteration to one component of the system). Other suitable strains may be, but are not limited to, Haematococcus pluvialis, Crypthecodinium cohnii, Neochloris oleoabundans, Schyzochytrium limacinum, Scenedesmus sp., Scenedesmus obliquus, Chlorella minutissima, Chlorella protothecoides, Chlamydomonas reinhardtii, Nitzschia laevis, Phaeodactylum tricornutum, Aurantiochytrium limacinum, Chlorella zofingiensis, Chlorella pyrenoidosa, Chlorella sp., Chlorella saccharophila, Chlorella sorokiniana, Galdieria sulphuraria, Dunaliella sp., Euglena gracilis, Nannochloropsis oculata, Nitzschia alba, Prototheca zopfii, Scenedesmus acutus, Schizochytrium sp., Schizochytrium sp., Brachiomonas submarina, Dunaliella tertiolecta, Tetraselmis verrucosa, Tetraselmis suecica, Tetraselmis tetrathele, Poterioochromonas malhamensis, Dunaliella salina, and Chlorella regularis.

After growing the Chlorella, the resulting biomass is harvested from AGC 152A. The remaining effluent contains less organic carbon and suspended solids than in the original brewery waste input stream, which allows light penetration in the effluent. Since a heterotrophic algal strain such as Chlorella, when grown heterotrophically, is less efficient in utilizing nitrogen and phosphorus, a majority of the nitrogen and phosphorus in the input stream to AGC 152A remains along with several micro-nutrients such as Ca, Mg, Na, Fe, Zn S, etc. Thus, in this experiment, the second effluent (resulting from the first system after separating biomass) composition is as follows:

Suspended solids (TSS) mg/L 47.95

Total C of TSS % dry wt. 1.66

Total N mg/L 10.3

NH4-N mg/L 0.03

Orth-phosphate mg/L 14.9Total P mg/L 16.1

K mg/L 53.6Ca mg/L 16.6

Mg mg/L 7.96

Na mg/L 175

Fe mg/L 0.16

Zn mg/L 0.04

S mg/L 32.00

As is notable, there is less organic carbon left in this effluent, which is to be expected based upon the selection of Chlorella in AGC 152A, and a significant amount of nitrogen and phosphorus also remain. Accordingly, the algal strain for AGC 152B was chosen so as to be able to capitalize on availability of non-organic carbon from CO2 gas (an off-gas of AGC 152A), and the ammonium, nitrogen, and phosphorus in the effluent coming from AGC 152A. For this experiment, the algal strain Scenedesmus was chosen, which requires inorganic carbon (CO2) to grow and does a better job at removing nitrogen and ammonium than does the heterotrophic Chlorella. For the phototrophic system, additional considerations for system design include, but are not limited to, CO2 level, the concentration of solids, light penetration, and the depth of growth system. Other suitable phototrophic strains could be Botryococcus braunii, Chlorella sp., C. minutissima, C. vulgaris, C. pyrenoidosa, Spirulina sp., Dunaliella sp., Hematococus sp., Schizochytrium sp., Nitzschia sp., N. dissipata, N. palea, Boekelovia hooglandii, Monallantus salina, Navicula sp., S. rophila, N. acceptata, N. pelliculosa, N. pseudotenelloides, Dunaliella Sp., Neochloris oleoabundans, Monoraphidium sp., Amphora Ourococcus, Nannochloris sp., Nannochloropsis salina, Scenedesmus sp., Scenedesmus obliquus, Scenedesmus quadricauda, Scenedesmus caudata, Scenedesmus bijunga, Desmodesmus sp., Ankitodesmus Chaetoceros sp., C. calcitrans, C. muelleri, Cyclotella cryptica, Amphiprora hyalina, Cylindrotheca sp., Pavlova lutheri, Amphora, E. oleoabundans, A. falcatus, C. sorokiniana T suecica, Lygnbya sp., Spirogyra sp., Ulothrix sp., Microspora sp., Claophora sp., Melosira sp., Lygnbya sp., Aphanocapsa sp., Asterionella sp., Navicula sp., Stephanodiscus sp., Tabellaria sp., Micractinium sp., Actinastrum sp., Rhizoclonium hieroglyphicum, Microspora willeana L, Ulothrix ozonata Kütz, Rhizoclonium hieroglyphicum Kütz, Oedogonium sp., Euglena sp., Chlamydomonas sp., Oscillatoria sp., etc. Although certain strains listed above for the second algal growth component are also listed from the first algal growth component, it is understood that the same strain would not be used for both the first and second algal growth components.

At a high level, the algal selection process is focused on the inputs to the given algal growth component, e.g., AGC 152A. Once the inputs are known (generally through laboratory analysis), the algal strain that provides the desired outcome, e.g., biomass production, removal of an entrained element, contamination reduction, etc., is chosen. As is understood by a person of ordinary skill in the art, suitable algal strains may be found (or made) from published research and databases, bioprospecting, or genetic engineering. Generally, a small-scale pilot is conducted prior to full system implementation to confirm the algal strain growth rates, its ability to meet outcome expectations, to determine the byproducts after growth, and to determine the existence (or lack thereof) of unexpected outputs, e.g., growth inhibiting compounds, that may need to be removed or destroyed (via, for example, heating up the effluent) before feeding the output to, for example, AGC 152B. Notably, even if the selected algal strain produces a toxin or growth inhibiting compound, an algal strain can be selected that will grow in the presence of that compound or toxin. See Harris, D. O., Growth Inhibitors Produced by the Green Algae (Volvoaceae), Arch Mikrobiol 76, 47-50 (1971). In any event, it would be understood by a person of ordinary skill in the art that a number of suitable algae strains would be available for pairing with a given input which would result in algal strain growth and would produce an output that would be pairable with another algal strain.

The algal system described herein may include a detector (or multiple detectors) to detect and provide information regarding the growing conditions in AGC 152A and 152B, and may further include one or more controllers to automatically shut down or start-up operation of AGC 152A and 152B in response to the information provided by the detector. Parameters (growing conditions) that may be monitored include, but are not limited to, the amount of light, the temperature, pH, and the presence (or absence) of contamination.

The algal system may include one or more detectors (sensors) and/or controllers which sense conditions in AGC 152A and/or 152B and alert a user to conditions that are favorable or unfavorable for growth. For example, the phototrophic reactor may be equipped with a sensor that measures or senses the quantity or amount of sunlight or other light (e.g. in lumens) that impinges on the phototrophic culture at any given time, or the cumulative amount of sunlight that impinges on the phototrophic culture during a period of time of interest (e.g. during a minute, and hour, a day, etc.). Detectors included with the algal system may be designed so as to provide information to a user whereby a user can decide whether to continue phototrophic culture, or to discontinue (stop, cease, shut down) phototrophic culture and harvest the resulting biomass. Although a heterotrophic bioreactor does not utilize sunlight, it may be situated in a manner that allows a sensor of relevant conditions to be associated therewith such as dissolved oxygen, pH, temperature, etc., and to record information and provide information that can be used to adjust conditions in the bioreactor to improve growing conditions.

In addition to sensing light levels, or instead of sensing light, the detector may be designed to also monitor (track, sense, measure, etc.) other parameters, including but not limited to temperature, the presence of contaminants, or in response to conditions in one or both of the reactors (e.g. when the heterotrophic reactor is inoperable due to maintenance, malfunction, etc.). Those of ordinary skill in the art will recognize that many useful parameters may be monitored, all of which are encompassed by the invention. Further, such detectors and controllers may be used to fine-tune the reactors, e.g. to detect and inform a user of the status (e.g. of growth rate) in the heterotrophic reactor so as to “ramp up” (or down) the level of activity in the phototrophic reactor, or vice versa, and may control feed rates of any inputs to one or both of AGC 152A and AGC 152B. For example, CO2 delivery to AGC 152B may be monitored so as to determine whether enough CO2 is coming from AGC 152A or if supplemental CO2 is needed (which can, for example, be provided by other facility operations, such as the exhaust from a combustible heating system). In other words, modulation of the activity of the two reactors in response to sensors need not be all or nothing, but can be implemented by degrees, either manually or automatically. In fact, when multiple reactors are employed, the controllers may be equipped to determine how many of the reactors are operable at a given time.

In another embodiment the size of algal core 156 can be deduced inversely, e.g., first the maximum amount of biomass producible via AGC 152B on the site is determined (usually space/volume limited) based upon the type of algal system, inputs, and space/footprint available, then the CO2 the requirements of the AGC 152B are determined, which can then be used to determine the composition and size of AGC 152A.

In yet another exemplary embodiment of algal core 156, an oxygen rich air supply from AGC 152A (when implemented as a photobioreactor as a result of photosynthesis by photoautotrophic or mixotrophic algae) is fed into AGC 152B (when implemented as a heterotrophic reactor to support growth of heterotrophic algae). This arrangement solves a major well-known constraint in closed photobioreactor systems caused by excessive oxygen production which has an adverse effect on the algae growth inside the photobioreactor.

In a further embodiment, an AGC 152A feeds AGC 152B while AGC 152B feeds AGC 152. For example, AGC 152A may feed CO2 to AGC 152B, while AGC 152B, concomitantly, feeds O2 to AGC 152B. Additional CO2 or O2 can be fed to the respective components for additional biomass production and carbon capture as desired.

Of the many advantages offered by SAS 100 and specifically by algal core 156, is the scalable nature of the system. Scalability is enhanced because heterotrophic algae (i.e., AGC 152A) is capable of dense growth when compared to photoautotrophic algae and certain mixotrophic algae. While density allows for greater biomass production per volume, heterotrophic algal growth in AGC 152A produces an off-gas, CO2 and effluent containing nitrogen, phosphorus, and other components requiring treatment before discharge. However, the need and concomitant expense of treatment can be mitigated (or even eliminated in certain embodiments) by incorporating AGC 152B because the second algal growth component uses the CO2 and effluent created by the AGC 152A, thus significantly reducing waste treatment costs while producing additional algal biomass.

While algal core 156 has been described above as a part of a larger system, e.g., SAS 100, algal culturing system 108, etc., it can also be implemented as a standalone system.

As shown in FIG.3, an algal core 200 can also use post algal harvest liquid effluent obtained from AGC 204A as an input for AGC 204B so as to provide an additional supply of nutrients.

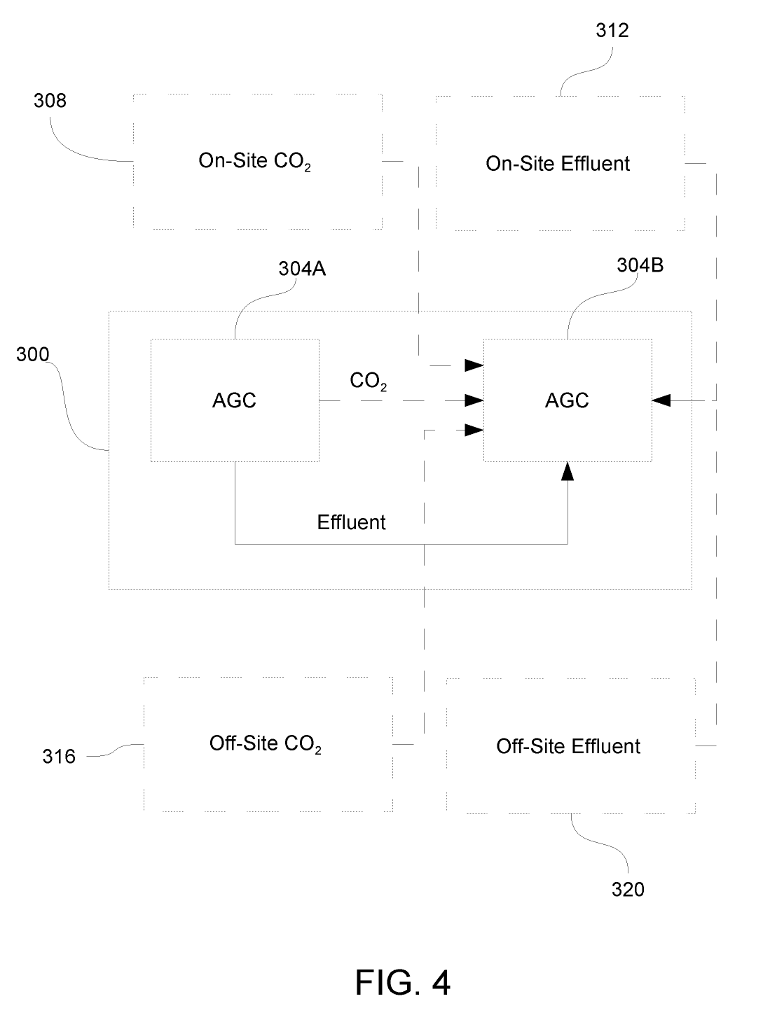

In yet another embodiment of algal core 300, and as shown in FIG. 4, a first AGC provides nutrients, but little if any (optionally) CO2 to a second AGC. This embodiment may be useful at sites where other means of CO2 capture, e.g., fossil fuel emissions capture, are available. Advantageously, using an algal core of this embodiment may also assist a CO2 emitting facility keep CO2 emissions within emission limits as the excess CO2 can be fed to one of the AGCs.

Another embodiment of algal core, algal core 300, is shown in FIG. 4. In this embodiment, algal core 300 includes a pair of AGCs, AGC 304A and 304B. AGC 304B is optionally fed with various sources of CO2 sources from either on-site resource 308, off-site resource 312, or from AGC 304A, or combinations of two or more of these CO2 sources. For example, at a dairy farm, the anaerobically digested effluent containing nitrogen and phosphorus is an on-site resource. The supplementary nutrient source from off-site could be the effluent from a creamery, cheese factory etc. FIG. 4 also shows AGC 304B being fed with additional sources of nutrients from either on-site resource 308 or off-site resource 320, including, but not limited to industrial waste, brewery waste and/or surplus, food waste and/or surplus, farm waste and/or surplus, and/or municipal waste.

Returning now to a discussion of FIG. 1, algal harvesting system 112 is used to collect the algal biomass generated by algal culturing system 108 when the algae is mature or considered ready for harvesting for biomass processing. In an exemplary embodiment, algal harvesting system 112 includes one or more solid separators 160, e.g., solid separator 160A and 160B, and a nutrient tank 164. Whether or not the output of AGC 152A or 152B should be sent to a solid separator 160 is determined by the type of output produced by the AGC. In an exemplary embodiment, and as shown in FIG. 1, AGC 152A produces a relatively low concentration algal biomass and thus solid separator 160A is used to concentrate the output of the AGC. In contrast, in an exemplary embodiment, AGC 152B produces a relatively concentrated algal biomass output that can be sent directly to a biomass processor 168 (described in more detail below).

When algal harvesting system 112 is in use, algae biomass from AGC 152A is provided to solid separator 160A, which in this embodiment is a settling tank that allows the algae mass to settle to the bottom of the tank. In this embodiment, the bottom quarter of the settling tank (or so) is then physically separated from the rest of the settling tank's contents. The top three quarters of the settling tank (generally a liquid layer) is pumped out of solid separator 160A (and can be re-fed into either AGC 152A or AGC 152B, or sent to algal biomass processing system 116, as discussed below) leaving only the bottom algae concentrate which can be subsequently removed.

Algal solids (also referred to as concentrate) separated out by algal harvesting system 112 are sent to algal biomass processing system 116, which can be a standalone unit or a combination of centrifugation, filtration, drying, gravity settling, microbial or chemical based biomass aggregation, flocculation and sedimentation, etc., to further concentrate the algal solids. As shown in FIG. 1, algal biomass processing system 116 includes a pair of biomass processors 168 (biomass processors 168A and 168B). In an embodiment, at least one biomass processor 168 is implemented as a separation funnel tank equipped with electrodes. In this embodiment, the algae concentrate from algal harvesting system 112 is gravity fed into the separation funnel tank. A current is then run through the algal concentrate, via the electrodes, causing individual algae cells to burst thereby releasing the lipids inside. The mixture within the separation funnel tank can then be allowed to separate into three layers, a solid layer (also referred to as “cake” layer), a water layer, and a lipid layer. The separation funnel tank can then be used to individually remove each layer for further processing or use. In another exemplary embodiment, algal biomass processing system 116 harvests algae from man-made water collection structure such as tanks, pits, ponds, etc., or natural water bodies such as ponds, tributaries, lakes, etc. in addition to being a part of SAS 100. The harvested algae can be become part of the algae cake and/or processed for different byproducts production such as fertilizer. In exemplary embodiments, algal biomass processing system 116 is implemented as a centrifuge, or as a unit that is immersed or floats on water to harvest biomass. For instance, a biomass processing system 116 can be installed at a farm that has nutrient runoff collection pits installed, which captures farm runoff and thereby naturally produce additional algae and microbes. A biomass processing system 116 can harvest these algae and microbes and add them to the algae cake.

In another embodiment, algal biomass processing system 116 is implemented as a solid separator that includes a centrifuge and a filtration system (e.g., filtration 170, shown in FIG. 1) so as to further reduce the amount of phosphorus and phosphate remaining in the effluent. In this embodiment, after separation of the algal biomass using a solid separator, the resulting effluent is further treated using the filtration system. The filtration system can a include one or more of a carbon-based material, such as, but not limited to, char, ash, and biochar of any size, texture, shape or composition. The filtration system can also include non-carbon materials such as, but not limited to, sand, gravel, limestone, zeolite, dolomite, mineral, clay, natural iron-based, and/or natural aluminum-based compounds. The filtration system may also include a barrier such as, but not limited to, geotextile, fabric, plastic, latex, fiberglass, metallic, earthen, and cemented, or combinations of the aforementioned. The filtration system can be implemented by various methodologies, such as, but not limited to, a tank, a bag, a sock, a pillow, a tube, a tote, a plate, a container, of any shape, size, or volume, and multiple filtration systems can be layered or suspended in a container. In operation, the effluent is allowed to flow-through filtration system and/or the filtration system can be suspended in the effluent. In certain embodiments, the filtration system includes an aerator so as to aerate the effluent during filtration.

A non-limiting example of an example of an algal biomass processing system 116 including a filtration system is now presented. As algae is known to utilize from over 50% to 99% of phosphorus, the inclusion of a filtration system increases the removal of phosphorus from the effluent stream and produces a useful byproduct. In this example, manure waste was sent used in the symbiotic algae system, e.g., SAS 100. After growth of the algae in AGCs 152A and 152B, the resulting algal biomass was separated with a centrifuge. Analysis of the resulting effluent determined that the algae had removed 95.45% of the phosphorus and 98.99% of phosphate from the manure waste. The resulting effluent was then provided to one of three different filtration systems. Filtration system No. 1 consisted of ash material, which resulted in 99.93% of the phosphorus being removed and about 100% of the phosphate. Filtration system No. 2 consisted of biochar made from wood, which also resulted in 99.93% of phosphorus being removed and about 100% of phosphate. Filtration system No. 3 comprised a layered system including biochar, ash, and sand. This system resulted in the removal of between about 99.94% to 99.99% of phosphorus and about 100% of phosphate. The resulting liquid from each of the filtration systems discussed above had reduced dissolved organic material, and the carbon-based material (e.g. ash, biochar) after filtration was activated. Advantageously, the resulting carbon-based material can be included with other materials to create a soil amendment and/or fertilizer. In certain embodiments, the resulting carbon-based materials is added to the algae cake.

Although the filtration system described above is included with algal biomass processing system 116, the filtration system can be included in other stages of SAS 100 as well. For example, the filtration system can be included with waste nutrient preparation sub-system 104 as an effluent pretreatment unit that outputs liquid waste, with less total phosphorus and phosphates to algal culturing system 108. In an embodiment, separator 136 includes the filtration system. In another embodiment, the filtration system is included with algal culturing system or algal harvesting system 112, whereby the algae growth container or the settling tank, respectively, includes the filtration system.

Algae cake with or without the addition of wild or naturally occurring algae or the carbon-based materials from the filtration system can be dried or mixed with additional biomass for conversion into byproducts such as, but not limited to, fertilizers and biofuels. In some of the instances of biomass processing system 116, the algae cake is densified by the addition of a secondary material or a mix of materials such as sawdust, hay, grasses, pelletization or pucks waste or surplus, lumber waste or surplus, wood waste, or surplus, etc. These densification processes may be beneficial to the renewable diesel production processes described below, to the formation of a storable form of fertilizer, or for the creation of combustible algal pellets for burning in gasifiers for heat. In some instances, algae cake alone or mixed with one or several materials, as described above, is pelletized or prepared into pucks, briquettes, pellets, etc., thereby providing increased storability. In another embodiment, algae cake is mixed with grasses grown on wasteland, or in buffer zones for capturing nutrients, e.g., miscanthus, switchgrass, etc., and/or with the carbon-based materials from the filtration system and then is formed into pellets, briquettes, pucks, etc.

Byproducts system 120 further treats the outputs received from algal biomass processing system 116. In an exemplary embodiment, from the lipid layer, crude algae oil is extracted with a solvent and a catalyst through a suitable process (chemical or non-chemical) at biofuel processor 172 so as to produce biodiesel and glycerol. In another exemplary embodiment, algae cake is converted into different forms of marketable fertilizer (either or both liquid and solid types). The solid fertilizer can be made into different forms such as powder, granular, pelleted, etc. and can include different proportions of nitrogen, phosphorus, and potassium (commonly combined and referred to as N—P—K). Producing algae fertilizer with marketable N—P—K concentrations has proved elusive. However, in certain embodiments of SAS 100, different algae types (monocultures, polycultures, or aggregations of naturally occurring algae with or without other microbes or components), capable of capturing different fertilizer constituents (e.g., N, P, K), are grown separately either in the looped reactor or in combined or standalone autotrophic, mixotrophic, or heterotrophic reactors or open ponds. Harvested algae can then be mixed in different proportions to obtain the marketable equivalent compositions of N—P—K, for example, as in Alfalfa meal (N—P—K: 2-1-2); Soymeal (7-2-1); and chicken manure (1.1-0.8-0.5). Algae fertilizer can also be enhanced by blends of different commercially or locally available materials, for example, by adding trace minerals for creating algae-based seed starting mixes, or by adding potassium for creating certain desirable N—P—K composition. Granular fertilizer can be made using fertilizer processor 176, which, in an exemplary embodiment is a commercially available granulating machine. In an exemplary embodiment, algal cake with sufficient moisture is dried prior to granulation. It has been reported that solid form of fertilizer applications improves crop growth by providing the captured nutrients in a relatively stable and storable form, which is not possible with the application of liquid manure on the land via manure spreader. This inefficiency exists because there are only few time windows available for liquid manure spreading during the crop growth. However, using a storable, granulated form of algal-based fertilizer provides flexibility of application during the times when a manure spreader cannot be used, such as for dressing the corn plants at the appropriate stage of their development. An environmental benefit, among others, of removal of nutrients via algal fertilizer is the reduction of nutrient runoff into natural water bodies. Moreover, cost offsets would be economically beneficial as fertilizer production produces an income stream for the farms or other businesses.

In yet another embodiment of byproducts system 120, biofuel processor 172 can convert algal biomass directly from algal culturing system 108, or through algal harvesting system 112, or algal biomass processing system 116 into “renewable diesel” and byproducts via hydrogenation (treatment with addition of hydrogen) via processes such as, but not limited to: a) hydrothermal processing (for instance, by reacting the biomass on the order of 15 to 30 minutes in water at a very high temperature, typically 570° to 660° F. and pressure 100 to 170 atm standard atmosphere, enough to keep the water in a liquid state to form oils and residual solids); b) indirect liquefaction (for instance, a two-step process to produce ultra-low sulfur diesel by first converting the biomass to a syngas, a gaseous mixture rich in hydrogen and carbon monoxide, followed by catalytic conversion to liquids, the production of liquids is accomplished using Fischer-Tropsch (FT) synthesis as applied to coal, natural gas, and heavy oils; c) integrated catalytic thermochemical process such as integrated hydropyrolysis and hydroconversion (IH2); d) hydroprocessing (the hydrothermal liquefaction (HTL) of biomass provides a direct pathway for liquid biocrude production via two types of methods possible for conversion of fatty acids to renewable diesel: “high-pressure liquefaction” or “atmospheric pressure fast pyrolysis”).

Potable fresh water is produced as a byproduct of algal harvesting system 112 that can be recycled for other uses.

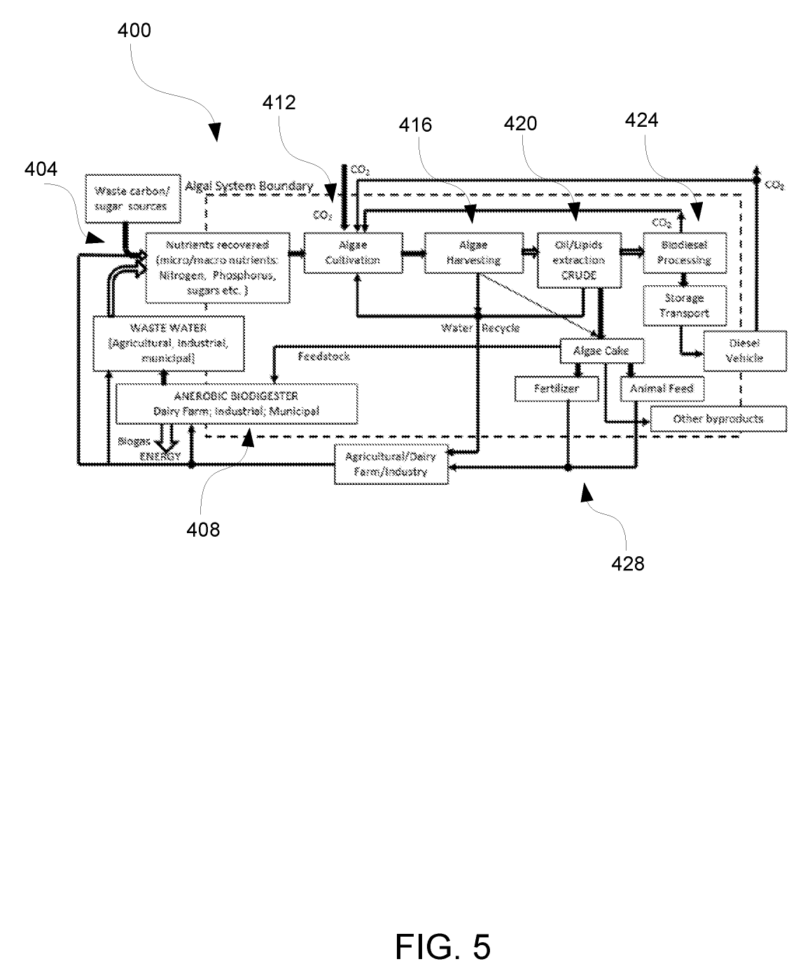

Turning now to FIG. 5, there is shown another exemplary symbiotic algae system, SAS 400, according to an embodiment of the present disclosure. At a high level, SAS 400 includes, but is not limited to, acquiring of feedstock inputs 404 from, for example, stakeholders, pretreater 408, algae cultivator 412, biomass harvester 416, oil extractor 420, byproducts manufacturer 424, and recycling of materials 428. Feedstock inputs 404 may be from a variety of stakeholders external to the SAS 400 operators, such as dairy manure waste generated at a farm (or in case of an industrial process, such as brewery, its generated wastes). Feedstock inputs 404 are processed through pretreater 408, which can be an anaerobic digester that, in addition to generating effluent useful for algae cultivation, can also generate biogas and/or bio-electricity as alternative energy. Pretreater 408 is capable of generating an effluent/wastewater stream with reduced odor and biochemical oxygen demand (BOD), which is advantageous for water quality. However, typically the pretreatment process of pretreater 408 does not remove nitrogen and phosphorus, which is a significant environmental issue, and due to government regulations typically requires further treatment for its safe discharge into natural water bodies. SAS 400 recovers the nutrients from effluent/wastewater via algae cultivator 412, which can be, for example, an embodiment of algal culturing system 108 as described herein. At a desired time, the algal biomass produced by algae cultivator 412 is harvested at biomass harvester 416, which outputs lipids, water, and solids—each of which can be a useful product or recycled within SAS 400. For example, lipids are extracted at oil extractor 420; water can be recycled into one or more of the other processes within SAS 400 such as algae cultivator 412 or back to one of the stakeholders (such as a dairy farm); and solids can be converted into animal feeds or fertilizers. The post-harvest algal biomass (also referred to as algae cake), and/or other algal biomass is utilized for production of additional useful byproducts, such as fertilizer or animal feed depending on the throughput feedstock used. For example, algae biomass grown with dairy manure waste would be more appropriate as a fertilizer instead of animal feed due to required FDA compliance. In contrast, brewery effluent, which is a cleaner byproduct of beer processing and typically being food grade, can be used for producing algal biomass for high value animal feed. The crude oil extracted by oil extractor 420 goes through further processing to obtain desirable end products (biodiesel, oil-heat, jet fuel), and is then stored, transported, and used in vehicles, planes, or for heating purposes. Notably, as algae is a CO2 sink, one can expect that at least a portion of the CO2 generated by the local use of the aforementioned products can be recaptured by the algal biomass production process along with the CO2 from the farm operations. Heat captured by pretreater 408 or from other on-site operations can be used as a heat input for algae cultivator 412, biomass harvester 416, oil extractor 420, and/or for sterilizing the algae cake that is used for animal feed production.

### EXAMPLE 1

In this example, an algal core included a first algal growth component that was a heterotrophic component that included a heterotrophic algal strain and which generated and fed carbon dioxide to a second algal growth component was a photoautotrophic counterpart that included a photoautotrophic algal strain. It should be noted that the latter could be a photoautotrophic open pond/tank, or a hybrid system supporting photoautotrophic or mixotrophic growth.

Two sets of bioreactors were setup to represent a test (an embodiment of the algal core discussed above) and a control. The control system was a closed photobioreactor fed with ambient air. The test algal core included two closed reactors, a heterotrophic reactor and a photoautotrophic reactor (supporting heterotrophic and photoautotrophic algal growth, respectively), where the photobioreactor was connected to ambient air supply plus the additional carbon dioxide generated from the heterotrophic reactor produced as a result of a fermentation process. Both control and test systems were run in duplicate under the same temperature conditions, utilized artificially prepared media, and algae inoculums (also referred to as algae starter). In this experiment, when compared to the photoautotrophic counterpart, only half of the amount of algae starter was used in the heterotrophic reactor so as to maintain control over the heterotrophic reactor process.

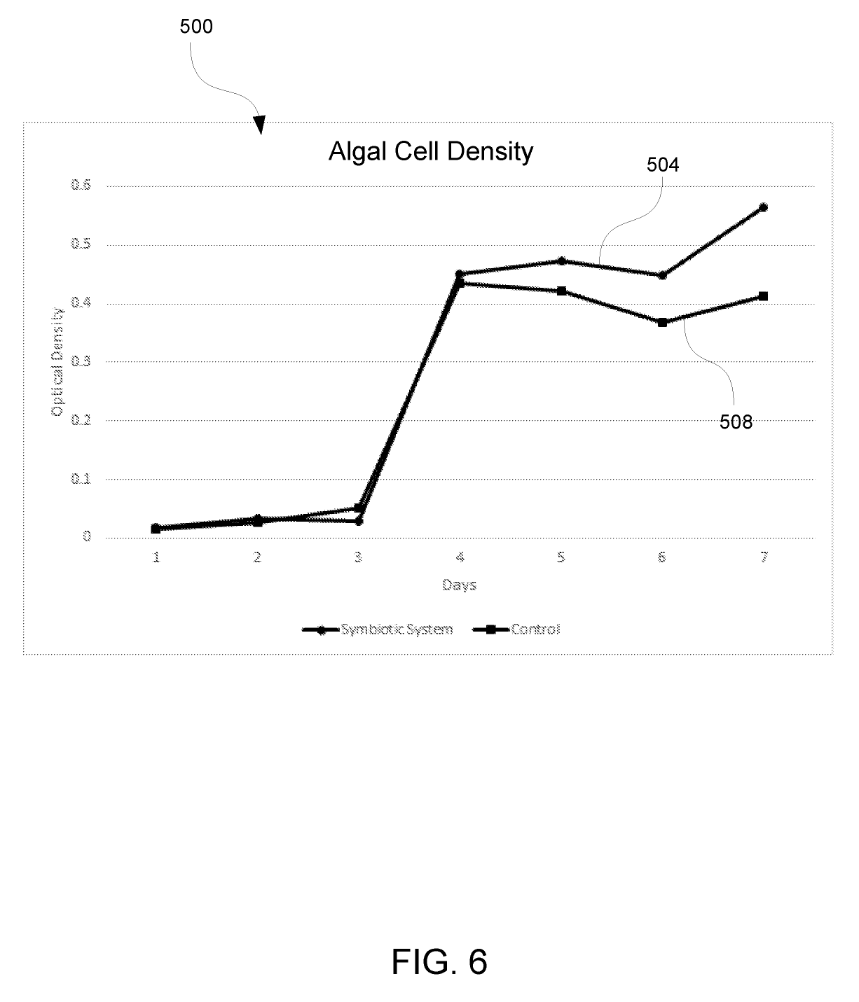

For the heterotrophic reactor, additional glucose was added to the artificial media, and the reactor was run without exposure to light. The photobioreactors had the same, constant light supply in both the test and the control batches. All reactors were regularly monitored for optical density, which indicates algal density (process discussed and shown in FIG. 6). Algal lipid content was monitored at the end of the log phase (day 4) and thereafter via confocal scanning laser microscope—a state-of-the-art multi-spectral imaging system using lipophilic dye. It was observed that the lipid content in the algal cells was negligible on day 4 and was highest on day 7 making it reasonable to harvest biomass on day 7. It should be noted that algae density can be strain and inoculum specific as some algae cultures may surpass the log phase earlier than 4 days, thereby making the harvest possible earlier than as shown in this example.

FIG. 6 shows a chart 500 of algae density (as measured by optical density) over time in days. Line 504 represents the test reactors and line 508 represents the control. As shown, very little algal density exists prior to day 4. After day 4, however, optical density substantially increases for both systems; however, algal density of the test system outpaces the control.

On the harvest day (day 7), the algal growth in the test algal core was found to be about 1.37 times higher (i.e., 37% more) than in the control reactor, which is considerable when extrapolated. For example, a typical, harvested photoautotrophic algae on dry weight basis is in the range of 300 mg/L (0.3 gm/L) to a 1 gm or more in photobioreactors. Using the more conservative harvest estimate, i.e., the 0.3 gm/L scenario, and extrapolating to an exemplary and typical 2000 ton/day algal growth system, a conventional photobioreactor system (or open pond system) would produce about 728,000 tons of algae biomass for oil extraction annually, whereas the photoautotrophic algal biomass harvest in the algal core, as discussed above, would be about 994,728 tons—a 266,728 ton surplus harvest.

As noted above, the heterotrophic reactor received 50% of the algae starter compared to the photobioreactor; however, if both reactors included an equal amount of algal inoculum, the amount of surplus algae from the heterotrophic reactor would be expected to double due to additional carbon dioxide generated by the heterotrophic reactor. If double the amount of heterotrophic algae was grown in the symbiotic system, this would contribute a surplus harvest of 3-4 times greater from the photobioreactor, thereby making the making the final surplus outcome about two or three times the harvest (i.e., about 74% to 111% more than the control). This example also illustrates how the volumes of the heterotrophic and photoautotrophic components in the symbiotic system could be customized to the algal harvest required from the two respective components. The surplus algal biomass generated could vary (lower or higher) in some embodiments depending on other factors such as media composition, light exposure, algae strain etc.

The examples and embodiments presented above could be applied to a variety of seed trains, where one system feeds a scaled-up version of the system. Various combinations of an SAS, such as SAS 100, could be made with the other existing algal growth systems and/or microbial growth systems.

### Looped Algae Reactor Design Pattern (LARDP)

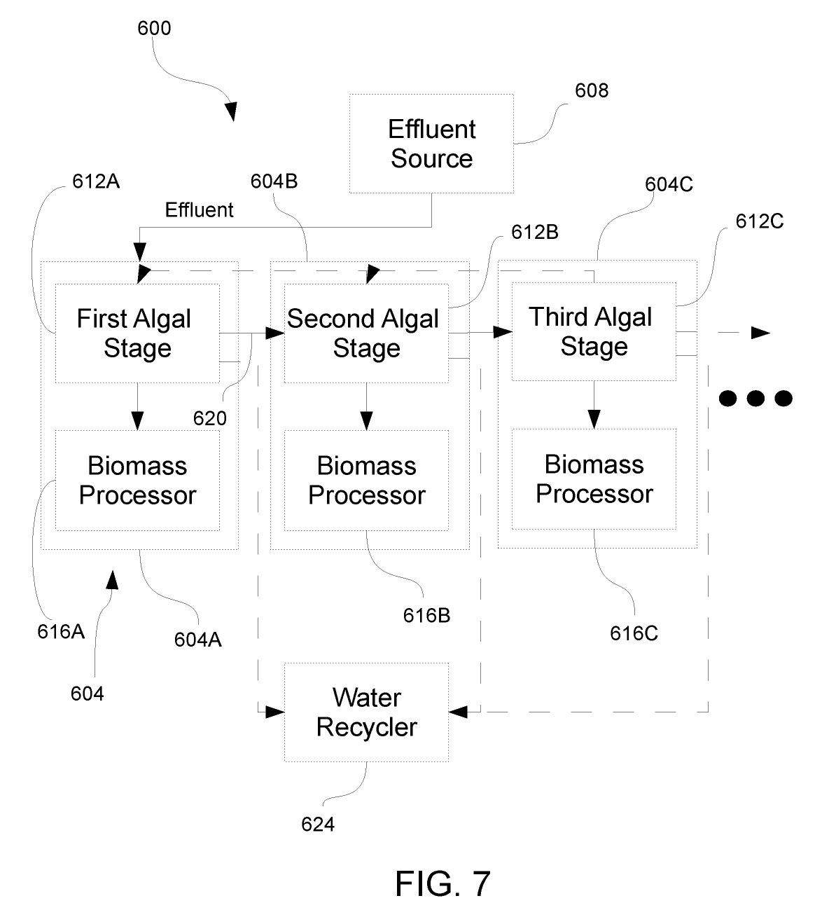

SAS 100 can, in certain embodiments, include a Looped Algae Reactor Design Pattern (LARDP) 600, as shown in FIG. 7. LARDP 600 is a process and/or a system that can be added/attached to algal cultering system 108, algal cores 156, 200, or 300, or can be a standalone system attached to a waste treatment, wastewater treatment, remediation system for cleaning wastewater/effluent streams using one or more strains of microalgae (or other microbial organisms such as bacteria, fungi etc.), or to any algae-based or microbial-based process producing a target product or byproducts. At a high level, LARDP 600 uses a process of repeated cultivation of algae for co-product development and/or removal of nutrients for improving water quality of the effluent stream by growing algae biomass with or without other microorganisms.

LARDP 600 can include a series of nutrient extraction systems (NES) 604, such as first NES 604A and second NES 604B. Each NES 604 is designed to extract a certain type or types of components from an incoming effluent stream 608, such as an algal effluent stream from an algal growth component, such as AGC 152A or 152B, or from other sources described herein. In an exemplary embodiment, first NES 604A includes a first algal stage 612A that receives an effluent stream as an input. First algal stage 612A is sized and configured to use microorganisms, such as those previously described herein, to extract from effluent stream 608 a certain type or types of components, such as, but not limited to, a nitrogen, a phosphorus, a heavy metal, a toxic component, a particular element (e.g. Ca, K, Mg, Na, Al, Fe, Mn, B, Cu, Zn, S, Pb, Cd, As), a complex element such as an antioxidant (e.g. astaxanthin), and a nuclear component. First algal stage 612A allows for the growth of the microorganisms and, in certain embodiments, can be similar in design to AGC 152B. At a desired time, the algal biomass produced by first algal stage 612A is harvested at biomass processor 616A, which can be performed as described above. First algal stage 612A also produces an effluent 620, which is at least partially devoid of the component that first algal stage 612A was designed to remove. This effluent can proceed to one or more primary pathways. The effluent can 1) be recirculated back to first algal stage 612A for further extraction of components (not shown), 2) proceed to a water recycling unit 624 for further water treatment, 3) proceed to second algal stage NES 604B, and/or 6) return to algal culturing system 108 (FIG. 1) (when LARDP 600 is coupled to such a system). In general, the concentration of the dominating component in the effluent 620 determines its destination. For example, if first algal stage 612A contained predominantly the alga Chlorella vulgaris which removed certain amount of nitrogen and phosphorus such that effluent 620 contains almost no nitrogen but still contained phosphorus, the effluent would likely travel to the 612 system containing the microorganisms capable of utilizing phosphorus more efficiently than Chlorella. vulgaris, such as, but not limited to Oscillatoria sp.

Second NES 604B and third NES 604C can be sized and configured to remove the same or a different type of component than that removed form first NES 604A. Second NES 604B thus can similarly include, a second algal stage 612B and a biomass processor 616B, and similarly third NES 604C can include, a third algal stage 612C and a biomass processor 616C. Additional stages 604 can be included to further extract components from effluent streams and recirculation to each stage in place in LARDP 600 can be performed. For example, if at first NES 604A, a first heavy metal is removed such that after entering the first NES it is present in the effluent stream in a lower concentration, the effluent can proceed to second NES 604B where another component, for example, a second heavy metal is removed to a lower concentration. The effluent from second NES 604B can then be recirculated to the first NES 604A for further removal of the first heavy metal, which is facilitated by the lower concentration of the second heavy metal.

In another exemplary embodiment, LARDP 600 is sized and configured to produce organic fertilizer from effluent steam 608. In this embodiment, at each NES 604 a desired fertilizer component is removed, e.g., nitrogen, phosphorus, potassium, etc. As each NES 604 allows for the harvesting of a concentrated amount of the desired component that is entrained within the organism, e.g., algae, in the NES, specific and fairly pure amounts of the component can be harvested and then mixed together to obtain the desired fertilizer product.

In use, when attached to an algal system, such as algal culturing system 108, microalgae disposed within LARDP 600 is cultivated in the effluent generated by the algae growth system. In this embodiment LARDP 600 is designed to remove undesirable substances such as, but not limited to, unwanted nutrients (e.g., nitrogen and phosphorus) and heavy metals. The biomass resulting from LARDP 600 can then be harvested from the wastewater and, depending on what LARDP has been designed to extract, processed to produce useful products such as, but not limited to, fertilizer and compost, or can be used as feedstock for digesters producing energy such as biogas or bio-electricity. After removal of the undesirable substances as described above, the remaining wastewater can then further be treated by cultering the same or a similar strain of microalgae as used in algal growth system 108 for producing the primary product, or the remaining waster can be further treated by one or more different algae strain(s) used as a monoculture or a polyculture with or without other microorganisms such as bacteria or fungi to further remove nutrients (e.g., nitrogen and phosphorus) or heavy metals or any other undesirable components present in the wastewater generated. LARDP 600 can be repeated in one or more stages with same or different strains of algae and/or bacteria and/or fungi or any other organisms compatible with algal strains, grown as a monoculture or polyculture in any type of algal growth system until the desired level of water quality is reached.

The number of NES 604s used in LARDP is determined by the number of desired removable elements in the effluent(s) that require capturing using microalgae or microbes and the desired water quality.

In an embodiment of the system, the one or more 604 stages in LARDP can be optionally combined or replaced by other processes such as multiple screening systems, decanting centrifugation, polymer flocculation, ammonia stripping, struvite formation, nitrification/de-nitrification, etc. Modifications of these processes can also be used for enhancing the whole process of nutrient removal.

LARDP 600 can be useful in the creation of products, including, but not limited, to biofuels, fertilizer, animal feed, and cosmetics. The organisms cultivated in LARDP 600 can be cultivated under a green house or other similarly enclosed environment, so as to prevent contamination by competitive microorganisms while admitting light. LARDP 600 can be implemented in, for example, vertical freestanding tanks, raceway style ponds, or tracks.

Additional useful byproducts from SAS 100 include the production of clean carbon dioxide (as compared to the CO2 captured from flue gases) generated from an algal growth component, such as AGC 152A, which, while discussed previously as supporting AGC 152B, can also be captured and used for other applications needing a clean source of CO2, e.g., medical applications, electronics, laboratories, etc. Alternatively, the CO2 can be used for algal inoculum-preparation (a highly concentrated algae culture typically used for seeding a larger scale system), especially to generate light-dependent inoculum for seeding a system or sub-system.

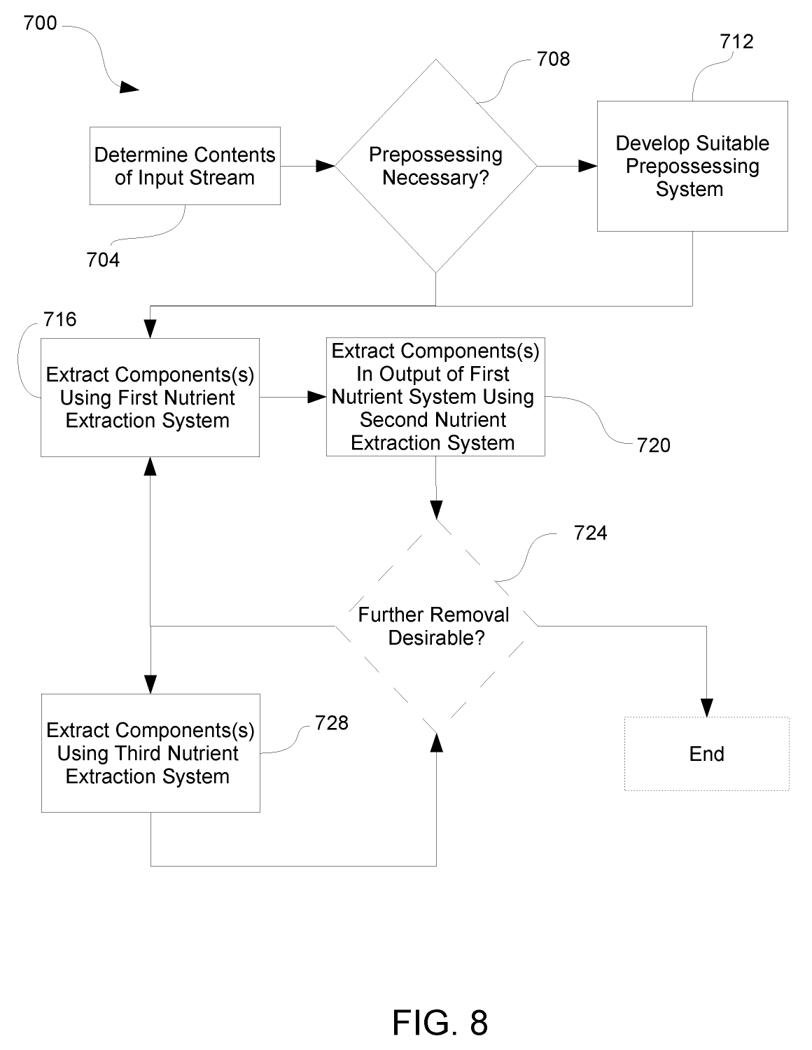

FIG. 8 shows a process 700 for removing contaminants from a waste or effluent stream. At step 704, the content of the waste or effluent stream is determined. While typical nutrients, such as nitrogen and phosphorus are likely to be found, the stream may also include heavy metals or other nutrients that are desirably removed from the stream before the stream is put to further use or otherwise treated. Determining which nutrients and other particles are a part of the waste or effluent stream will assist in determining the type of nutrient extraction system, such as one of the NES 604s discussed above, to implement.

At step 708 it is determined whether any preprocessing is necessary prior to the stream entering the first NES. Preprocessing may be necessary if the stream contains significant solids or too much liquid. If preprocessing is necessary, process 700 proceeds to step 712 where a suitable preprocessing system is developed. Exemplary preprocessing systems are solids treatment unit 140 and separator 136 as discussed above with reference to FIG. 1. If no preprocessing is necessary, process 700 proceeds to step 716.

At step 716, a first NES is used to extract components from the waste or effluent stream. In an exemplary embodiment, first NES is sized and configured to focus on a relatively small number of components for extraction. For example, if the input waste or effluent stream is nitrogen rich, first NES may be configured to include an algal component that is primarily effective at removing a substantial portion of the nitrogen from the waste or effluent stream. The output of first NES is then provided to a second NES at step 720 for extraction of another component of the original waste or effluent stream.

At optional step 724, a determination is made as to whether further removal of nutrients from the output of step 720 is desired. As part of step 724 a determination of the composition of the output of step 720 may be completed so as to determine where, if anywhere, the output of step 720 should be sent. For example, in order to effectively remove a heavy metal from a waste stream, it is generally beneficial to remove nutrients that are in the stream in significant amounts. Therefore, if, for example, the output of step 720 included significant amounts of a nutrient, e.g., nitrogen, that would render extraction of the heavy metal difficult or inefficient, step 724 would determine that the stream should be sent to an NES that will efficiently remove more nitrogen (e.g., step 716). However, if removal of a different component is desired, process 700 may proceed to step 728 where a third NES is used to extract components form the output stream. If no further extractions are necessary, the process ends.

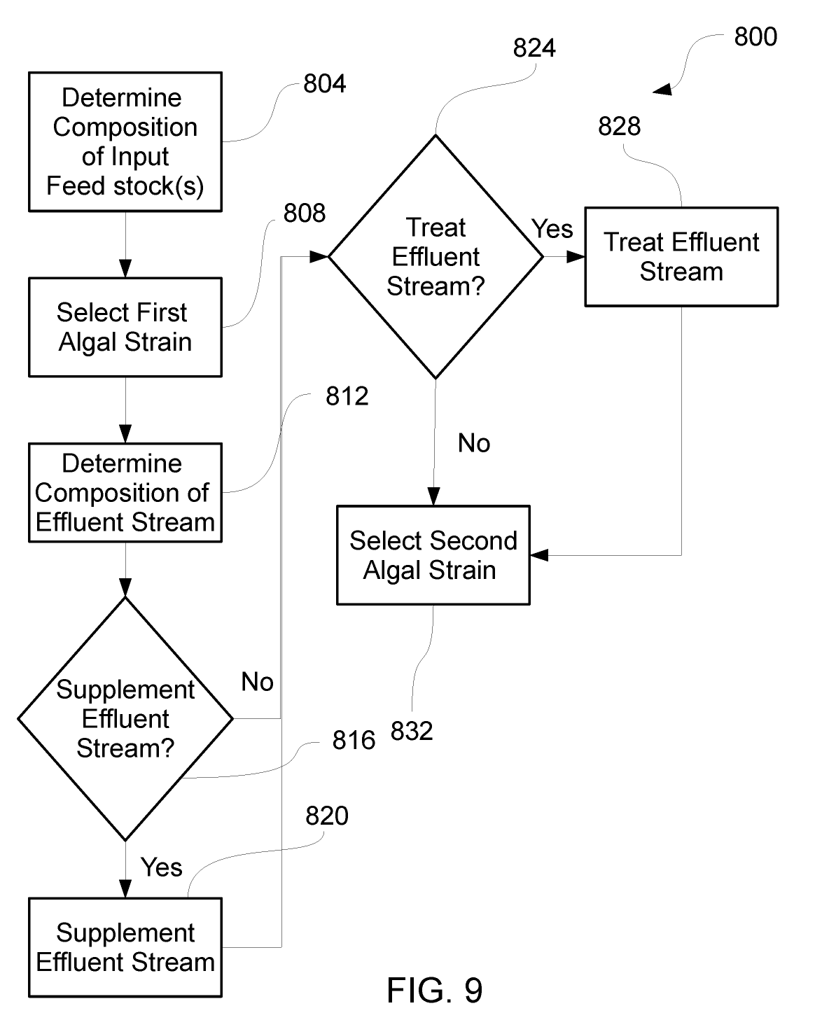

FIG. 9 shows a process 800 of selecting an algal strain for AGC 152A and AGC 152B (or for any algal growth component that is part of the system). At step 804, the input stream(s) that are expected to be delivered to AGC 152A are evaluated for the composition of the input steam(s). Typical input streams have been described elsewhere herein, such as, brewery waste, manure, and the like. These input streams may be pretreated to prepare for delivery to the algal growth component. Additional input streams may include a recycled stream from the second algal growth component. If the recycled stream is included, the evaluation may need to be iterative because the selection of the first and second algal strains impacts the composition of the recycled stream.

At step 808, the first algal strain is selected for algal growth component 152A. The selection is based on the composition of the input stream and the desired result from the growth of the algae, e.g., biomass production, reduction of an entrained element, heavy metal removal, etc. The growth characteristics of a given algal strain may be known from published research or may be understood through small-batch evaluation of algae that is bioprospected or genetically engineered.

At step 812, the composition of the effluent stream is evaluated so as to determine the components in the stream.

At step 816, a determination is made as to whether the effluent stream needs to be supplemented with additional nutrients to promote growth. For example, in the brewery waste example above, the effluent was low in organic carbon. If the system designer desires to use an algal strain that needs some organic carbon for growth, a supplemental source may be provided at step 820.

If no supplement is needed or if the supplement has been provided, the process proceeds to step 824 where a decision is made as to whether to treat the effluent stream. The effluent stream may need treatment depending on its contents, the desired output of the entire system, and the desired algal strain that is used in algal growth component 152B. For example, if the effluent includes a growth inhibiting component, and, while a suitable algal strain can be used, another strain would produce a more desirable outcome but would be partially inhibited by the contents of the effluent, the effluent can be treated. In a preferred embodiment, the selection of the first and second algal strains for AGC 152A and 152B, respectively, are chosen so as to not inhibit the growth of either strain so as to avoid system complexity.

If it is desired to treat the effluent stream, the process proceeds to step 828. If no treatment is desired, the process proceeds to step 832.

As is evident, many of the steps of process 800 are dependent on other steps as well as the desires of the system designer.

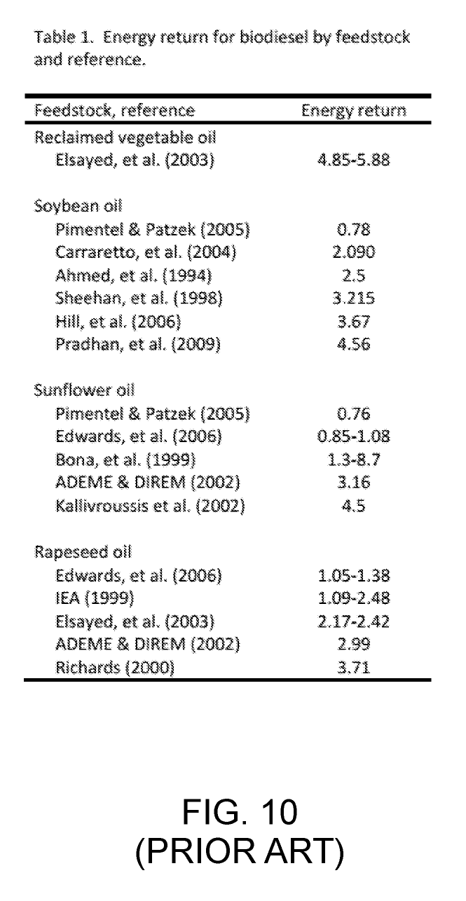

Turning now to a discussion of FIG. 10, there is shown energy return values (EROI) for biodiesel by feedstock. The EROI is calculated as the ratio between the energy produced and the energy consumed by a system, and is generally considered a critical measure for evaluating the net energy profitability of that system. As the EROI increases, the energetic profitability of that energy system also increases. For any feedstock (e.g., algae, soybean oil, etc.) or combination of feedstocks (e.g., SAS 100) to be a net energy source, the EROI to operate the entire associated production system(s) must be greater than 1. However, historically, the EROI of viable energy sources has been much greater than 1 and, therefore, practical deployment of an energy source typically requires an EROI much greater than 1. For instance, the EROI has been used to characterize several conventional fuels; for example, for coal, oil and gas, and corn ethanol, the second-order EROI has been estimated to be ˜80 (at the mine), ˜15 (at the well), and ˜1 (at the biorefinery). Delivered gasoline (considering the entire supply chain) has reported an overall EROI of around 5 to 10.

As shown in FIG. 10, the EROI range is a low of 0.76 for sunflower oil to a high of about 5.88 for reclaimed vegetable oil. EROI for a typical phototrophic algae system (considered to be the lowest cost algae production method) is between 1 and 3. For a heterotrophic algae system, the EROI is much lower (due to the cost of the system). Thus, it would not be expected that adding an expensive heterotrophic system in series with a phototrophic system would result in an EROI. However, certain embodiments of SAS 100 (varying pretreatment and algae types) have obtained energy return values of about 1, 11, and 40. The primary reason for the large jump in EROI is the mutually beneficial coupling between the two growth systems, which reduces feedstock costs.

In an exemplary aspect, a symbiotic algae system is disclosed that comprises: a first algal growth component, wherein the first algal growth component includes a heterotrophic organism, and wherein the first algal growth component produces a first effluent and an off-gas; and a second algal growth component is fluidly coupled to the first algal growth component, and the second algal growth component including at least one organism from the group of: a photoautotrophic organism, a mixotrophic organism, and a heterotrophic organism, and wherein the second algal growth component receives, as an input, the first effluent and the off-gas and produces a second effluent. In the symbiotic algae system, the first algal growth component can receive, as a first input, an effluent input or a waste input. In the symbiotic algae system, the second algal growth component can receive, as a second input, an effluent input or a waste input. The symbiotic algae system can further include a waste nutrient preparation sub-system fluidly coupled to the first algal growth component. In the symbiotic algae system, the waste nutrient preparation sub-system can receive an effluent input, a fresh water input, and waste input, and outputs an effluent suitable for use by the first algal growth component. In the symbiotic algae system, the waste nutrient preparation sub-system is a manure settling and solids preparation unit that outputs liquid manure waste to the first algal growth component. The symbiotic algae system can further include an algal harvesting system having at least one separator, wherein the algal harvesting system is fluidly coupled to the first algal growth component and/or the second algal growth component. The symbiotic algae system can have an EROI greater than 10. The symbiotic algae system can have an EROI of about 40. The symbiotic algae system can further comprise a third algal growth component, wherein the third algal growth component includes at least one organism from the group of: a photoautotrophic organism, a mixotrophic organism, and a heterotrophic organism, and wherein the third algal growth component receives, as an input, the second effluent. The symbiotic algae system can further comprise at least one biomass processing system, the biomass processing system sized and configured to extract lipids from at least one of the first algal growth component and the second algal growth component.

In another exemplary aspect, a symbiotic algae system is disclosed that comprises a first algal growth component, wherein the first algal growth component includes a heterotrophic organism, and wherein the first algal growth component produces a first effluent and an off-gas; and a second algal growth component fluidly coupled to the first algal growth component, wherein the second algal growth component includes at least one organism from the group of: a photoautotrophic organism, a mixotrophic organism, and a heterotrophic organism, and wherein the second algal growth component receives, as a first input, the first effluent and the first off-gas and produces a second effluent and a second off-gas; and wherein the second effluent and the second off-gas are received as inputs to the first algal growth component. In the symbiotic algae system, the first algal growth component can receive, as an additional input, an effluent input or a waste input, and wherein the additional input and the second effluent include a nitrogen and a phosphorous. In the symbiotic algae system, the first algal component can remove a portion of the nitrogen and the phosphorus from the second input and the additional input. The symbiotic algae system can further comprise a third algal growth component, wherein the third algal growth component includes at least one organism from the group of: a photoautotrophic organism, a mixotrophic organism, and a heterotrophic organism, and wherein the third algal growth component receives a portion of the second effluent. The symbiotic algae system can further comprise at least one biomass processing system, the biomass processing system sized and configured to extract lipid/oil from at least one of the first algal growth component and the second algal growth component. The symbiotic algae system can have an EROI greater than 10. The symbiotic algae system can have an EROI of about 40.

In yet another exemplary aspect, a symbiotic algae system can comprise: a waste nutrient preparation sub-system; an algal culturing system including: a first algal growth component fluidly coupled to said waste-nutrient preparation sub-system, wherein the first algal growth component includes a heterotrophic organism, and wherein the first algal growth component produces a first effluent and an off-gas; and a second algal growth component, wherein the second algal growth component includes at least one organism from the group of: a photoautotrophic organism, a mixotrophic organism, and a heterotrophic organism, and wherein the second algal growth component receives, as an input, the effluent and the off-gas and produces a second effluent; and an algal harvesting system fluidly coupled to said algal culturing system; an algal biomass processing system fluidly coupled to said algal harvesting system; and a byproducts system fluidly coupled to said algal biomass processing system. In the symbiotic algae system, the waste nutrient preparation sub-system can receive, as an input, an effluent input or a waste input.

In an embodiment, a symbiotic algae system for removing nutrients from an input stream including organic carbon waste matter that is either phosphorus rich or nitrogen rich, the symbiotic algae system comprises a first algal growth component, wherein the first algal growth component includes a first container suitable for growing a first heterotrophic algae strain, and wherein the first algal growth component produces a first algal biomass, a first effluent including a portion of the organic carbon waste matter and a first growth byproduct, and a carbon dioxide rich off-gas; and a second algal growth component fluidly coupled to the first algal growth component, the second algal growth component including a second container suitable for growing a second algal strain, wherein the second algal strain is a different strain than the first heterotrophic algae strain, and wherein the second algal growth component produces a second algal biomass, a second effluent including a smaller portion of the organic carbon waste matter and a second growth byproduct, and an oxygen rich off-gas, a first algal harvesting system coupled to the first container and a second algal harvesting system coupled to the second container, wherein the first algal harvesting system separates the first algal biomass from the first effluent and the second algal harvesting system separates the second algal biomass from the second effluent, and wherein the second algal growth component receives, as an input, the first effluent, after removal of the first algal biomass, and the carbon dioxide rich off-gas, and wherein the first algal growth component receives the second effluent, after removal of the second algal biomass, and the oxygen rich off-gas, and wherein the first heterotrophic algae strain is selected based upon the input stream, and wherein the second algal strain is selected based upon the first effluent. Additionally or alternatively, wherein the first algal growth component does not receive any oxygen from any other source besides second algal growth component. Additionally or alternatively, wherein the second algal growth component does not receive any carbon dioxide from any other source besides first algal growth component. Additionally or alternatively, wherein the second algal growth component receives, as a second input, a waste input, and wherein the algal strain is selected based upon the first effluent and the waste input. Additionally or alternatively, wherein the first algal biomass is not harvested until the first heterotrophic algal strain is mature. Additionally or alternatively, wherein the system has an EROI greater than 10. Additionally or alternatively, wherein the system has an EROI of about 40. Additionally or alternatively, wherein the symbiotic algae system further comprises at least one biomass processing system coupled to the first and/or second algal harvesting system, the biomass processing system sized and configured to extract one or more of a lipid, a protein, a carbohydrate, a metabolite, and a dye, from the first algal biomass and/or the second algal biomass.

In another embodiment, a symbiotic algae system for removing nutrients from an input stream including organic carbon waste matter that is either phosphorus rich or nitrogen rich, the symbiotic algae system comprises a first algal growth component, wherein the first algal growth component includes a first container suitable for growing a first heterotrophic algae strain, and wherein the first algal growth component produces a first algal biomass, a first effluent including a portion of the organic carbon waste matter and a first growth byproduct, and a carbon dioxide rich off-gas; and a second algal growth component fluidly coupled to the first algal growth component, the second algal growth component including a second container suitable for growing a second algal strain, wherein the second algal strain is a different strain than the first heterotrophic algae strain, and wherein the second algal growth component produces a second algal biomass, a second effluent including a smaller portion of the organic carbon waste matter and a second growth byproduct, and an oxygen rich off-gas, and wherein the second algal growth system receives, as an input, the first effluent and the carbon dioxide rich off-gas wherein the first algal growth system receives the second effluent, and wherein the first heterotrophic algae strain is selected based upon the input stream and the second effluent, and wherein the second algal strain is selected based upon the first effluent. Additionally or alternatively, wherein the first algal strain also receives the oxygen rich off-gas. Additionally or alternatively, wherein the first algal growth component does not receive any oxygen from any other source besides second algal growth component. Additionally or alternatively, wherein the second algal growth component does not receive any carbon dioxide from any other source besides first algal growth component. Additionally or alternatively, wherein the symbiotic algae system further includes a first algal harvesting system coupled to the first container and a second algal harvesting system coupled to the second container, wherein the first algal harvesting system separates the first algal biomass from the first effluent and the second algal harvesting system separates the second algal biomass from the second effluent. Additionally or alternatively, wherein the symbiotic algae system further comprises at least one biomass processing system coupled to the first and/or second algal harvesting component, the biomass processing system sized and configured to extract lipids from the first algal biomass and/or the second algal biomass. Additionally or alternatively, wherein the second effluent is not further treated or processed prior to delivery to first algal growth component. Additionally or alternatively, wherein the second algal growth component receives, as a second input, a waste input, and wherein the algal strain is selected based upon the first effluent and the waste input. Additionally or alternatively, wherein the first algal biomass is not harvested until the first heterotrophic algal strain is mature. Additionally or alternatively, wherein the system has an EROI greater than 10.

A symbiotic algae system according to claim 9, wherein the system has an EROI of about 40.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A symbiotic algae system for removing nutrients from an input stream including organic carbon waste matter and which is either phosphorus rich or nitrogen rich, the symbiotic algae system comprising:
a first algal growth component, wherein the first algal growth component includes a first container suitable for growing a first heterotrophic algae strain, and wherein the first algal growth component produces a first algal biomass, a first effluent including a portion of the organic carbon waste matter and a first growth byproduct, and a carbon dioxide rich off-gas; and
a second algal growth component fluidly coupled to the first algal growth component, the second algal growth component including a second container suitable for growing a second algal strain, wherein the second algal strain is a phototrophic or mixotrophic algal strain that is different strain than the first heterotrophic algae strain, and wherein the second algal growth component produces a second algal biomass, a second effluent including a smaller portion of the organic carbon waste matter, a second growth byproduct, and an oxygen rich off-gas,
a first algal harvesting system coupled to the first container and a second algal harvesting system coupled to the second container, wherein the first algal harvesting system separates the first algal biomass from the first effluent and the second algal harvesting system separates the second algal biomass from the second effluent, and
wherein the second algal growth component receives, as an input, the first effluent, after removal of the first algal biomass, and also receives the carbon dioxide rich off-gas, and wherein the first algal growth component receives the second effluent, after removal of the second algal biomass, and also receives the oxygen rich off-gas, and
wherein the first heterotrophic algae strain is selected based upon the input stream, and wherein the second algal strain is selected based upon the first effluent.

2. A symbiotic algae system according to claim 1, wherein the first algal growth component does not receive oxygen from any other source besides second algal growth component.

3. A symbiotic algae system according to claim 2, wherein the second algal growth component does not receive carbon dioxide from any other source besides first algal growth component.

4. A symbiotic algae system according to claim 1, wherein the second algal growth component receives, as a second input, a waste input, and wherein the second algal strain is selected based upon the first effluent and the waste input.

5. A symbiotic algae system according to claim 1, wherein the first algal biomass is not harvested until the first heterotrophic algal strain is mature.

6. A symbiotic algae system according to claim 1, wherein the system has an EROI greater than 10.

7. A symbiotic algae system according to claim 1, wherein the system has an EROI of about 40.

8. A symbiotic algae system according to claim 1, further comprising at least one biomass processing system coupled to the first and/or second algal harvesting system, the biomass processing system sized and configured to extract one or more of a lipid, a protein, a carbohydrate, a metabolite, a molecule, and a dye, from the first algal biomass and/or the second algal biomass.

9. A symbiotic algae system for removing nutrients from an input stream including organic carbon waste matter and which is either phosphorus rich or nitrogen rich, the symbiotic algae system comprising:
a first algal growth component, wherein the first algal growth component includes a first container suitable for growing a first heterotrophic algae strain, and wherein the first algal growth component produces a first algal biomass, a first effluent including a portion of the organic carbon waste matter and a first growth byproduct, and a carbon dioxide rich off-gas; and
a second algal growth component fluidly coupled to the first algal growth component, the second algal growth component including a second container suitable for growing a second algal strain, wherein the second algal strain is a phototrophic or mixotrophic algal strain that is a different strain than the first heterotrophic algae strain, and wherein the second algal growth component produces a second algal biomass, a second effluent including a smaller portion of the organic carbon waste matter and a second growth byproduct, and an oxygen rich off-gas, and
wherein the second algal growth system receives, as an input, the first effluent and the carbon dioxide rich off-gas,
wherein the first algal growth system receives the second effluent, and
wherein the first heterotrophic algae strain is selected based upon the input stream and the second effluent, and wherein the second algal strain is selected based upon the first effluent.

10. A symbiotic algae system according to claim 9, wherein the first algal strain also receives the oxygen rich off-gas.

11. A symbiotic algae system according to claim 10, wherein the first algal growth component does not receive oxygen from any other source besides second algal growth component.

12. A symbiotic algae system according to claim 11, wherein the second algal growth component does not receive carbon dioxide from any other source besides first algal growth component.

13. A symbiotic algae system according to claim 9, further including a first algal harvesting system coupled to the first container and a second algal harvesting system coupled to the second container, wherein the first algal harvesting system separates the first algal biomass from the first effluent and the second algal harvesting system separates the second algal biomass from the second effluent.

14. A symbiotic algae system according to claim 13, further comprising at least one biomass processing system coupled to the first and/or second algal harvesting system, the biomass processing system sized and configured to extract one or more of a lipid, a protein, a carbohydrate, a metabolite, and a dye, from the first algal biomass and/or the second algal biomass.

15. A symbiotic algae system according to claim 13, wherein the second effluent is not further treated or processed prior to delivery to first algal growth component.

16. A symbiotic algae system according to claim 9, wherein the second algal growth component receives, as a second input, a waste input, and wherein the second algal strain is selected based upon the first effluent and the waste input.

17. A symbiotic algae system according to claim 9, wherein the first algal biomass is not harvested until the first heterotrophic algal strain is mature.

18. A symbiotic algae system according to claim 9, wherein the system has an EROI greater than 10.

19. A symbiotic algae system according to claim 9, wherein the system has an EROI of about 40.

20. A symbiotic algae system for removing nutrients from an input stream including organic carbon waste matter and which is either phosphorus rich or nitrogen rich, the symbiotic algae system comprising:
a first algal growth component, wherein the first algal growth component includes a first container suitable for growing a first heterotrophic algae strain, and wherein the first algal growth component produces a first algal biomass, a first effluent including a portion of the organic carbon waste matter and a first growth byproduct, and a carbon dioxide rich off-gas;
a second algal growth component fluidly coupled to the first algal growth component, the second algal growth component including a second container suitable for growing a second algal strain, wherein the second algal strain is a phototrophic or mixotrophic algal strain that is a different strain than the first heterotrophic algae strain, and wherein the second algal growth component produces a second algal biomass, a second effluent including a smaller portion of the organic carbon waste matter and a second growth byproduct, and an oxygen rich off-gas; and
a filtration system fluidly coupled to the second algal growth container, the filtration system sized and configured to remove almost all the remaining non-water elements from the second effluent, the filtration system including a carbon-based material,
wherein the second algal growth system receives, as an input, the first effluent and the carbon dioxide rich off-gas, and
wherein the first heterotrophic algae strain receives the oxygen rich off-gas,
wherein the first heterotrophic algae strain is selected based upon the input stream, and wherein the second algal strain is selected based upon the first effluent, and
wherein the carbon-based material, after filtration of the second effluent, is suitable for inclusion with an algal based product.
